(19) United States
(12) Patent Application Publication
(10) Pub. No.: US 2005/0010213 A1
(43) Pub. Date: Jan. 13, 2005

(54) Attachment mechanism for surgical instrument

(75) Inventors: Shawn D. Stad, Fall River, MA (US); Christopher Rogers, Taunton, MA (US); Michael D. Sorrenti, Middleboro, MA (US); Patrick Fatyol, Whitman, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DePuy Spine, Inc., Raynham, MA (US)

(21) Appl. No.: 10/616,506

(22) Filed: Jul. 8, 2003

(51) Int. Cl. A61F 5/04

(52) U.S. Cl. 606/53

(57) ABSTRACT

An attachment mechanism for a surgical instrument is provided which includes a receiving component having a longitudinal axis, a modular tip configured to be coupled in a rigid manner to the receiving component in two or less orientations, and a locking mechanism for securing the tip to the receiving component. A method for attaching a modular tip to a surgical instrument is also provided that includes actuating a locking mechanism, inserting a mating component of a modular tip into a receiving component by moving the mating component in a direction substantially perpendicular to a longitudinal axis of the receiving component, or by rotating the mating component relative to the receiving component, or a combination thereof, and releasing the locking mechanism to secure the tip to the receiving component.

### BACKGROUND OF THE INVENTION

Surgical procedures, for example, orthopaedic and spinal orthopaedic procedures, are typically size-specific to account for anatomical differences in the patient population. As a result, instrumentation used to perform these procedures is designed to accommodate implants of various sizes and geometries. The ability of a surgical instrument to enable insertion of implants of differing sizes is important to the usability of the instrument and the overall success of the implantation. Surgical instruments having modular instrument components of various sizes and geometries have been previously proposed to allow the same instrument to carry out a wide variety of procedures, for example, compression and distraction of a patient's spine.

### SUMMARY OF THE INVENTION

A surgical instrument having a pair of handles is provided which includes a receiving component having a longitudinal axis, a modular tip configured to be coupled in a rigid manner to the receiving component in two or less orientations, and a locking mechanism for securing the tip to the receiving component. The tip can include a mating component configured to be coupled to the receiving component by insertion of the mating component into the receiving component in a direction substantially perpendicular to the longitudinal axis of the receiving component. Alternatively, or in addition to, the mating component can be configured to be coupled to the receiving component by rotating the mating component relative to the receiving component. A force applied to the mating component along the longitudinal axis of the receiving component in a direction away from the receiving component is resisted by the receiving component independently from any resistive force applied by the locking mechanism.

In a particular embodiment, the tip includes a mating component having at least three planar surfaces configured to engage at least three planar surfaces of the receiving component to resist a moment or load acting on the tip. In another embodiment, a conical surface of the mating component engages a conical surface of the receiving component (each surface being planar and tapered) when the tip is coupled to the receiving component. In this embodiment, two or more surfaces are engaged to resist a moment or load acting on the tip.

In one embodiment, the locking mechanism is configured to secure the mating component along at least two surfaces and can include a spring for resiliently biasing the locking mechanism in a locked position. The locking mechanism can include a first member and a second member that are moveable within the receiving component, with each member having a surface that engages, in a locked position, a corresponding surface of the mating component. In another embodiment, the locking mechanism does not include a spring and the first and second members are moveable by the user between locked and unlocked positions. In further embodiments, the locking mechanism can include a collar slideable along, or rotatable about, the receiving component.

The receiving component can include a recess and an opening that form a connecting member in the receiving component. In another embodiment, an opening in the receiving component forms the connecting member. The connecting member can be configured to cooperatively engage a recess in the mating component. In another embodiment, the receiving component can be provided at an end of a handle.

In particular embodiments, the surgical instrument can be used in the compression or distraction of objects, such as a patient's spine.

In other embodiments, an attachment mechanism for a device is provided which includes a modular tip including a mating component, a receiving component configured to be coupled in a rigid manner to the mating component in two or less orientations, and a locking mechanism for securing the mating component to the receiving component. The locking mechanism can include a collar slideable along, or rotatable about, the receiving component.

A method for attaching a modular tip to a surgical instrument is also provided in accordance with aspects of the invention. The method includes actuating a locking mechanism, inserting a mating component of a modular tip into a receiving component by moving the mating component in a direction substantially perpendicular to a longitudinal axis of the receiving component, or by rotating the mating component relative to the receiving component, or a combination thereof, and releasing the locking mechanism to secure the tip to the receiving component. The method can also include the step of detaching the tip from the receiving component by actuating the locking mechanism and removing the mating component from the receiving component by moving the mating component in a direction substantially perpendicular to the longitudinal axis of the receiving component.

### BRIEF DESCRIPTION OF THE DRAWINGS

The foregoing and other objects, features and advantages of the invention will be apparent from the following more particular description of various embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a perspective view of a surgical instrument in accordance with an embodiment of the invention.

FIG. 2 is an exploded view of an embodiment of an attachment mechanism in accordance with an embodiment of the invention.

FIG. 3 is a different perspective view of the attachment mechanism shown in FIG. 2.

FIG. 4 is a side view of the assembled attachment mechanism shown in FIGS. 2 and 3.

FIG. 5 is a cross-sectional view taken along line 5-5 of FIG. 4.

FIG. 6 is an exploded view of another embodiment of an attachment mechanism in accordance with other embodiments of the invention.

FIG. 7 is a perspective view of another embodiment of a locking mechanism in a locked position in accordance with another embodiment of the invention.

FIG. 8 is a perspective view of the locking mechanism of FIG. 7 in an unlocked position.

FIG. 9 is a perspective view of another embodiment of a locking mechanism in a locked position in accordance with yet another embodiment of the invention.

FIG. 10 is a perspective view of the locking mechanism of FIG. 9 in an unlocked position.

### DETAILED DESCRIPTION OF THE INVENTION

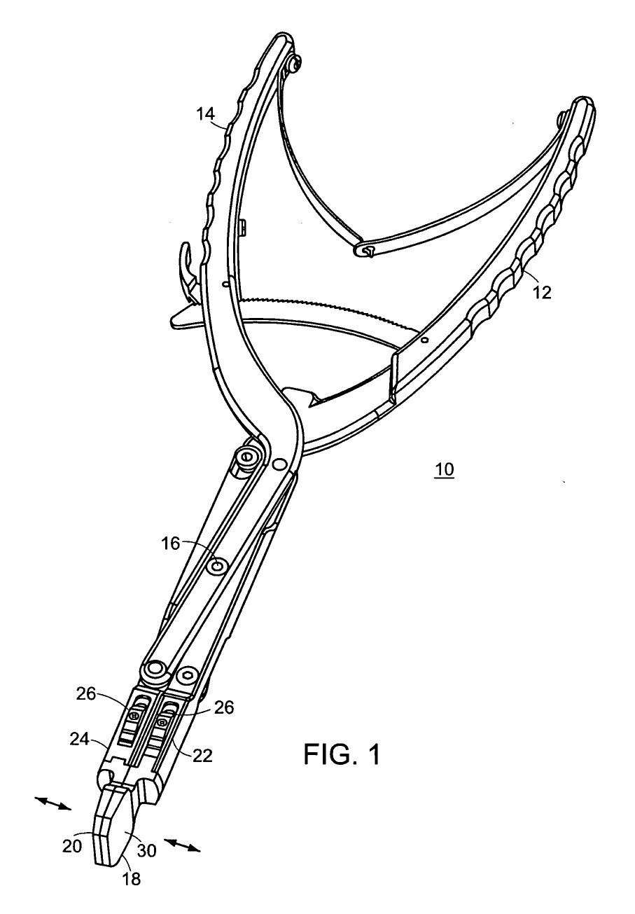

A description of various embodiments of the invention follows. FIG. 1 is a perspective view of a surgical instrument 10 provided in accordance with an embodiment of the invention. In the embodiment shown, the surgical instrument 10 can be referred to as a distractor for use in manipulation of a patient's spine. Handle members 12, 14 are pivotally connected by a pin 16 such that as the handles are squeezed together, the distance between tips 18, 20, supported by respective receiving components 22, 24, is increased to move the spine, for example.

The tips 18, 20 are secured to the receiving components 22, 24 in accordance with principles of the present invention. Applications, such as the distraction of two vertebral bodies, require an instrument that can apply a substantial amount of force. Additionally, parallel distraction requires both a great deal of mechanical advantage and a rigid instrument. Thus, an embodiment of an attachment mechanism used to affix tips 18, 20 to the receiving components 22, 24 is designed to provide a strong and rigid connection without sacrificing ease of use. In this embodiment, slideable members 26 are slid toward the handles 12, 14 to release the tips 18, 20 from the receiving components 22, 24, as will be discussed in more detail below. Components 22, 24 can be attached to respective handles 12, 14. In another embodiment, the receiving components 22, 24 can be integrally formed in the ends of handles 12, 14, i.e., machined into the end of the handles.

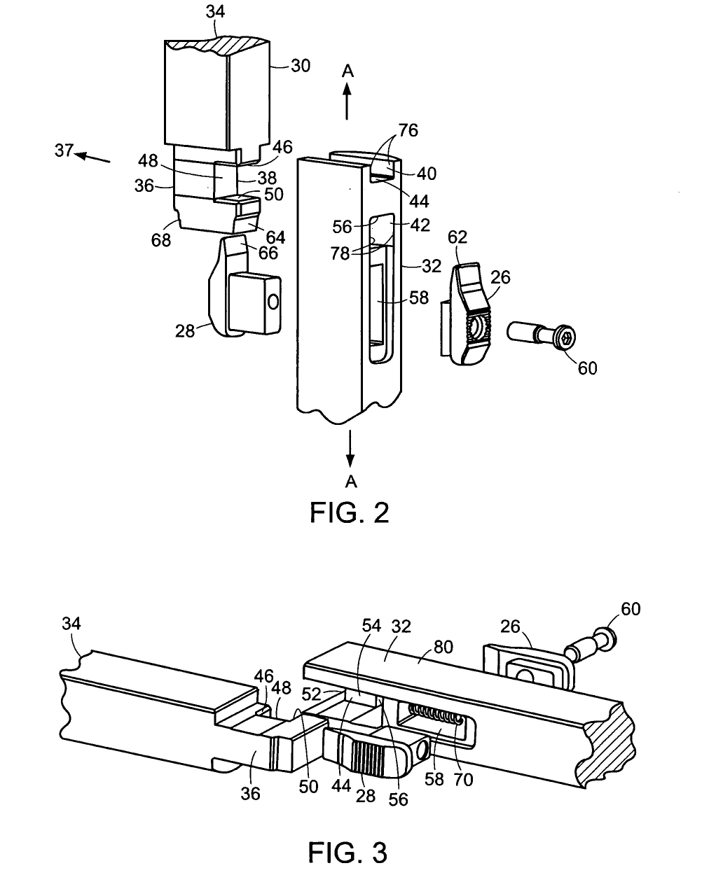

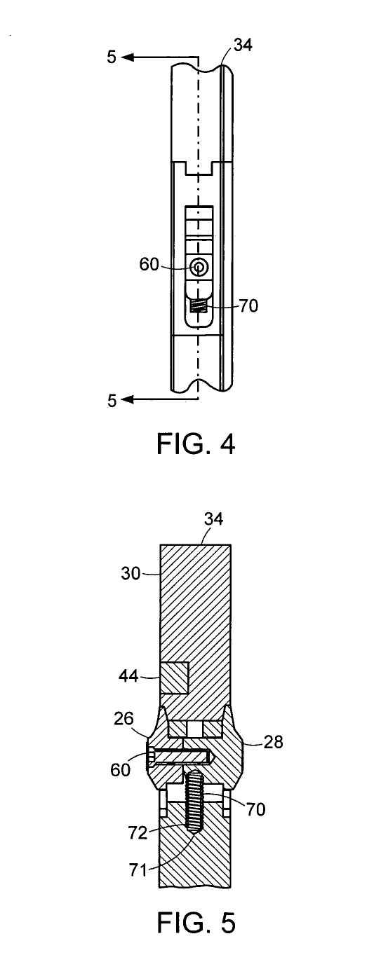

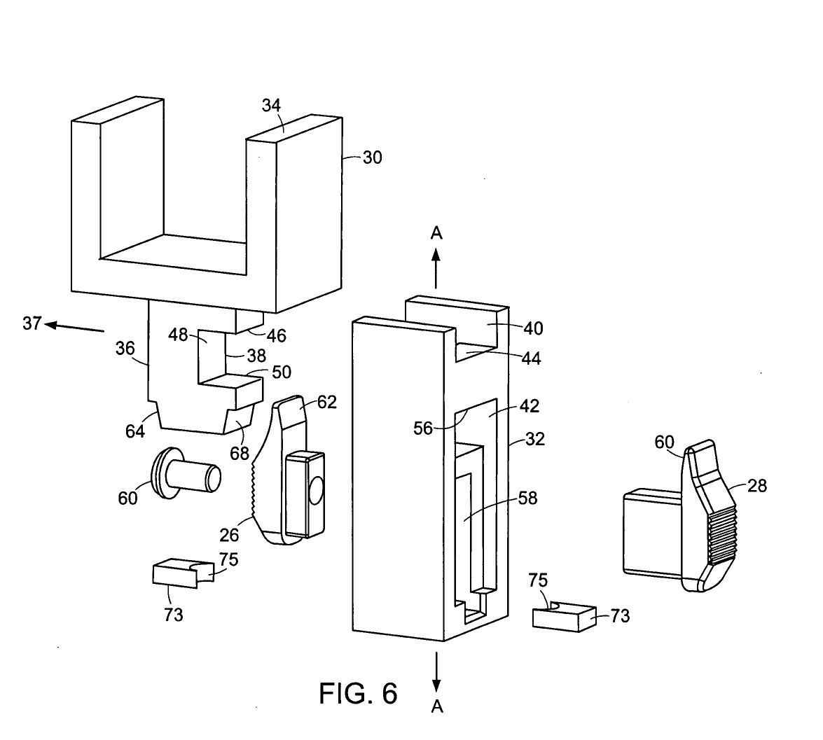

FIGS. 2-5 illustrate a modular tip 30 that is affixable to a receiving component 32 in accordance with an embodiment of the invention. In this embodiment, tip 30 has a an end 34, which can be U-shaped as illustrated in the embodiment of FIG. 6, although other geometries can be employed depending on the application. In this embodiment, a mating component 36 of the tip 30 is configured to be inserted into receiving component 32 in a single orientation. In one embodiment, an offset connecting member 44 formed by recess 40 and opening 42 is inserted into a recess 38 of the mating component 36. The connecting member 44 in receiving component 32 helps provide structural support to the component 32.

In another embodiment, the connecting member 44 is provided at the end of receiving component 32 such that the receiving component does not have recess 40. In an alternative embodiment, the connecting member 44 is provided at the center of the receiving component 32 such that the mating component 36 of tip 32 can be inserted into the receiving component 36 in two orientations.

Recess 38 can include a U-shaped cutout having three surfaces 46, 48, 50 configured to contact three surfaces 52, 54, 56 of connecting member 44 for maximizing the contact surface area between the mating component 36 and receiving component 32 to provide a strong and rigid connection between the mating and receiving components. The surfaces 46, 48, 50 and 52, 54, 56 are sufficiently tightly toleranced to provide the strong and rigid connection between mating component 36 and the receiving component 32 when the tip 30 is loaded by a force in the Cartesian directions and when loaded by a moment about any of the Cartesian axes. Thus, a force applied to the tip 30 and thus mating component 36 along the longitudinal axis A-A of the receiving component in a direction away from the receiving component, i.e., an axial force, is resisted by the receiving component 32 independently from any resistive force applied by the locking mechanism. That is, surfaces 46, 48, 50 of the mating component 36 engage surfaces 52, 54, 56 of the receiving component 32 to prevent relative movement between components 36, 32 unless a force is applied in a direction substantially perpendicular to the longitudinal axis A-A of the receiving component 32, i.e., in a direction along arrow 37. Surfaces 76 of recess 40 and surfaces 78 of opening 42 resist forces applied perpendicular to longitudinal axis A-A normal to surface 80 of receiving component 32.

In another embodiment, the connecting member 44 can be circular in cross-section and the recess 38 can be arcuate, i.e., the recess 38 forms a semi-circle. A hole can be drilled through the receiving component 36 and a rod can be inserted therein to provide the connecting member 44 that is configured to engage the semi-circular recess 38 in mating component 36.

Receiving component 32 can include a cavity 58 in which a locking mechanism for securing the mating component 36 to the receiving component 32 is movably disposed. In a particular embodiment, a first slideable member 26 is couplable to a second slideable member 28 and held in place by a fastener 60. First member 26 includes a surface 62 that engages, in a locked position, surface 64 of mating component 36. Similarly, second member 28 includes a surface 66 that engages, in a locked position, surface 68 of mating component 36. Thus, the locking mechanism is configured to secure the mating component along at least two surfaces thereof to provide a strong connection between the mating component 36 and the receiving component 32.

In one embodiment, a spring 70 resiliently biases the members 26, 28 in a locked position, i.e., toward the mating component 36. In another embodiment, the locking mechanism does not include a spring and the members 26, 28 are moveable by the user between locked and unlocked positions. A receiving hole 72 (FIG. 5) can be used to maintain the base 71 of spring 70 in position. As shown in the embodiment of FIG. 6, the base 71 of spring 70 can maintained in position by securing members 73 that are insertable into the receiving component 32. Each securing member 73 has a semi-circular shaped end 75 in this embodiment.

To release the tip 30 from the receiving component 32, the user slides members 26, 28 away from tip 30 and moves the tip in a direction along arrow 37.

The components of the device can be made of suitable materials that surgical instruments are typically made of For example, the material can include stainless steel, titanium, ceramic, plastic, or combinations thereof.

Although the embodiment shown in FIGS. 1-6 relates to a distractor, the same attachment mechanism can be used to attach modular tips to a compressor, or any other surgical instrument where changeable modular tips are desired. For example, the attachment mechanism can be used with implant insertion instruments, broaches, spine fusion instrumentation, and instruments used in arthroplasty procedures. Also, the same attachment mechanism can be used to attach interchangeable components where a strong and rigid connection is desired between a mating component and a receiving component.

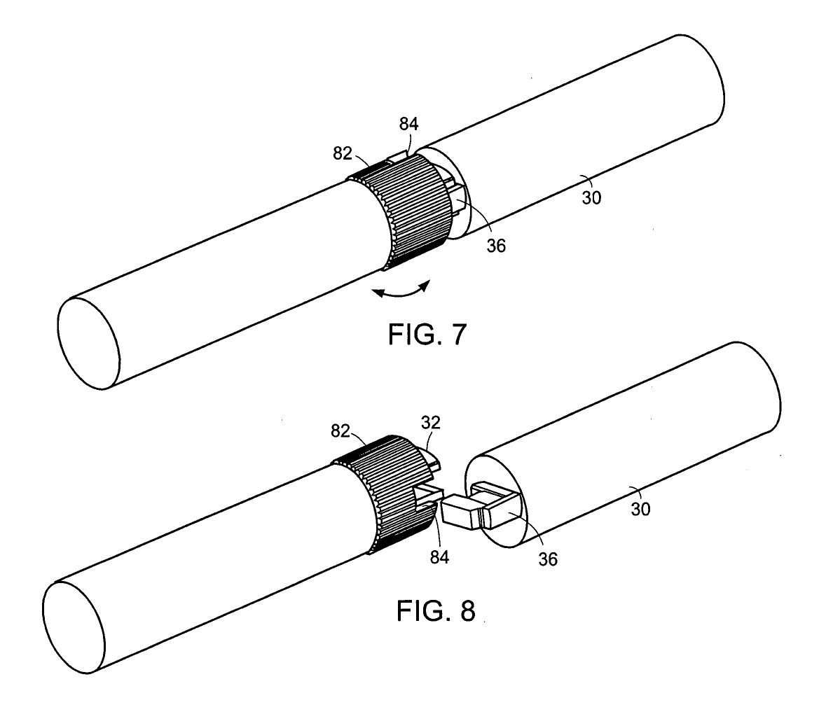

FIGS. 7 and 8 illustrate another embodiment of a locking mechanism used to secure a mating component 36 to a receiving component 32. In this embodiment, a collar 82 is rotatable about the receiving component 32. In one embodiment, a torsional spring can be used to bias in a locked position (FIG. 7). The collar 82 includes a slot 84 that allows the mating component 36 to be removed from the receiving component 32 when the user the collar 82 is rotated into an unlocked position as shown in FIG. 8.

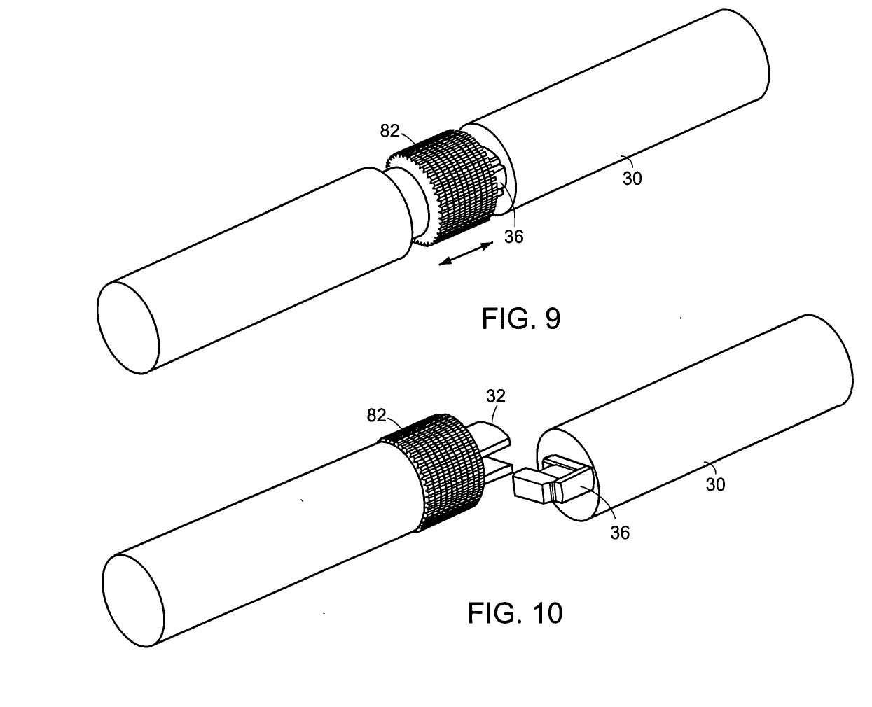

FIGS. 9 and 10 illustrate yet another embodiment of a locking mechanism used to secure a mating component 36 to a receiving component 32. In this embodiment, a collar 82 is slideable along the longitudinal axis of receiving component 32 between a locked position (FIG. 7) and an unlocked position (FIG. 8). In a particular embodiment, a spring can be used to resiliently bias the collar 82 in the locked position.

While this invention has been particularly shown and described with references to various embodiments thereof, it will be understood by those skilled in the art that various changes in form and details may be made therein without departing from the scope of the invention encompassed by the appended claims.

1. A surgical instrument comprising:
a receiving component having a longitudinal axis;
a modular tip configured to be coupled in a rigid manner to the receiving component in two or less orientations; and
a locking mechanism for securing the tip to the receiving component.

2. The surgical instrument of claim 1, wherein the tip includes a mating component configured to be coupled to the receiving component by insertion of the mating component into the receiving component in a direction substantially perpendicular to the longitudinal axis of the receiving component, or by rotating the mating component relative to the receiving component, or a combination thereof.

3. The surgical instrument of claim 1, wherein the tip includes a mating component configured to be coupled to the receiving component such that a force applied to the mating component along the longitudinal axis of the receiving component in a direction away from the receiving component is resisted by the receiving component independently from any resistive force applied by the locking mechanism.

4. The surgical instrument of claim 1, wherein the tip includes a mating component having at least three planar surfaces configured to engage at least three planar surfaces of the receiving component.

5. The surgical instrument of claim 1, wherein the tip includes a mating component having a conical surface configured to engage a conical surface of the receiving component.

6. The surgical instrument of claim 1, wherein the tip includes a mating component having planar surfaces configured to engage planar surfaces of the receiving component to resist a moment or load acting on the tip.

7. The surgical instrument of claim 1, wherein the locking mechanism is configured to secure the mating component along at least two surfaces.

8. The surgical instrument of claim 7, further comprising a spring for resiliently biasing the locking mechanism in a locked position.

9. The surgical instrument of claim 1, wherein the locking mechanism includes a first member and a second member being moveable within the receiving component, each member having a surface that engages, in a locked position, a corresponding surface of the mating component.

10. The surgical instrument of claim 1, wherein the locking mechanism includes a collar slideable along the longitudinal axis of the receiving component between a locked position and an unlocked position.

11. The surgical instrument of claim 1, wherein the locking mechanism includes a collar rotatable about the receiving component between a locked position and an unlocked position.

12. The surgical instrument of claim 1, wherein the tip includes a mating component configured to be coupled to the receiving component and wherein the receiving component includes a recess and an opening that form a connecting member in the receiving component, the connecting member being configured to cooperatively engage a recess in the mating component.

13. The surgical instrument of claim 1, wherein the tip includes a mating component configured to be coupled to the receiving component, further comprising a rod configured to cooperatively engage a semi-circular recess in the mating component.

14. The surgical instrument of claim 1, wherein the instrument is configured to be used in the compression or distraction of objects.

15. The surgical instrument of claim 1, wherein the receiving component is provided at an end of a handle.

16. A surgical instrument comprising:
a receiving component having a longitudinal axis;
a modular tip including a mating component configured to be coupled in a rigid manner to the receiving component; and
a locking mechanism for securing the mating component to the receiving component;
the mating component being coupled to the receiving component such that a force applied to the mating component along the longitudinal axis of the receiving component in a direction away from the receiving component is resisted by the receiving component independently from any resistive force applied by the locking mechanism.

17. The surgical instrument of claim 16, wherein the mating component is configured to be coupled to the receiving component by insertion of the mating component into the receiving component in a direction substantially perpendicular to the longitudinal axis of the receiving component, or by rotating the mating component relative to the receiving component, or a combination thereof.

18. The surgical instrument of claim 16, wherein the mating component includes planar surfaces configured to engage planar surfaces of the receiving component to resist a moment or load acting on the tip.

19. The surgical instrument of claim 16, wherein the planar surfaces are tapered.

20. The surgical instrument of claim 16, wherein the locking mechanism is configured to secure the mating component along at least two surfaces.

21. The surgical instrument of claim 16, wherein the receiving component includes a recess and an opening that form a connecting member in the receiving component, the connecting member being configured to cooperatively engage a recess in the mating component.

22. An attachment mechanism for a device, comprising:
a modular tip including a mating component;
a receiving component configured to be coupled in a rigid manner to the mating component in two or less orientations; and
a locking mechanism for securing the mating component to the receiving component.

23. The attachment mechanism of claim 22, wherein the locking mechanism includes a collar slideable along, or rotatable about, the receiving component.

24. A method for attaching a modular tip to a surgical instrument that includes a receiving component configured to be coupled to the tip, the instrument also including a locking mechanism for securing the tip to the receiving component, the method comprising:
actuating a locking mechanism;
inserting a mating component of the modular tip into the receiving component by moving the mating component in a direction substantially perpendicular to a longitudinal axis of the receiving component, or by rotating the mating component relative to the receiving component, or a combination thereof; and
releasing the locking mechanism to secure the tip to the receiving component.

25. The method of claim 24, further comprising the step of detaching the tip from the receiving component by actuating the locking mechanism and removing the mating component from the receiving component by moving the mating component in a direction substantially perpendicular to the longitudinal axis of the receiving component.
